(19) United States
(12) Patent Application Publication
(10) Pub. No.: US 2025/0072781 A1
(43) Pub. Date: Mar. 6, 2025

(54) MULTI-SENSOR DEVICE FOR MONITORING HEALTH

(71) Applicant: Analog Devices, Inc., Wilmington, MA (US)

(72) Inventors: Venugopal GOPINATHAN, Boston, MA (US); James Doscher, Exeter, NH (US); Tony J. Akl, Bedford, MA (US)

(21) Appl. No.: 18/951,164

(22) Filed: Nov. 18, 2024

Related U.S. Application Data
(63) Continuation of application No. 16/478,848, filed on Jul. 18, 2019.
(60) Provisional application No. 62/449,741, filed on Jan. 24, 2017.

(51) Int. Cl. A61B 5/0535; A61B 5/00; A61B 5/0205; A61B 5/332

(52) U.S. Cl. CPC A61B 5/0535; A61B 5/0205; A61B 5/332; A61B 5/6823; A61B 5/6831; A61B 5/684

(57) ABSTRACT

Devices, systems, and methods for non-invasively detecting and monitoring medical conditions using multiple modalities of sensing include at least two electrodes configured to be positioned on a subject, an acoustic sensor configured to be positioned on a subject, a thoracic impedance measurement module connected to the electrodes, for measuring a first impedance between the electrodes, and a heart acoustic measurement module connected to the acoustic sensor, for detecting and measuring a heart sound from the acoustic sensor.

### CROSS-REFERENCE TO RELATED APPLICATION

This application is a continuation of U.S. patent application Ser. No. 16/478,848, filed Jul. 18, 2019, which is a national stage application of International Patent Application No. PCT/US2018/015067, filed Jan. 24, 2018, which claims the benefit of and priority to U.S. Provisional Patent Application No. 62/449,741, filed Jan. 24, 2017, the contents of which applications are hereby incorporated by reference herein in their entireties.

### FIELD OF THE DISCLOSURE

The present application relates generally to systems, apparatus, and methods of managing medical or health conditions in human subjects, and more specifically to systems, apparatus, and methods of non-invasively detecting and monitoring medical or health conditions such as congestive heart failure (CHF) and Chronic Obstructive Pulmonary Disease (COPD) conditions in human subjects that employ multiple modalities of sensing.

BACKGROUND

In human subjects, congestive heart failure (CHF) is a known cardiac condition in which damaged heart muscle loses its ability to pump sufficient amounts of blood to meet the body's demands. In the early stages of CHF, such an inability to pump sufficient amounts of blood may occur only while a human subject exercises. However, in more advanced stages of CHF, such an inability to pump sufficient amounts of blood may occur even while the human subject is at rest. CHF is one of the most commonly diagnosed cardiac conditions in hospital patients over the age of 65, and one of the most frequent reasons for such patients' readmission to hospitals in a time duration of 30 days. In recent years, 30-day hospital readmission expenses for CHF have increased to $1.8 billion per year, with approximately $13,000 being allotted for each readmission at a 25% readmission rate. Some of the reasons for such patients' readmission to hospitals can include, but are not limited to, (1) patient non-compliance with regard to diet and medication, which can result in excess fluid in the lungs or extreme dehydration, (2) incomplete titration of medication dosages, which often need to be modified as a patient moves from the hospital environment back to his or her home, and (3) atrial fibrillation, which can onset after the patient's discharge from the hospital.

Management of CHF in patients following discharge from the hospital has traditionally focused on monitoring the patients' fluid retention using sensors incorporated in implantable cardiac devices, such as implantable cardioverter defibrillators (ICDs), cardiac resynchronization therapy-defibrillators (CRT-Ds), or pacemakers. Such implantable cardiac devices can detect developing pulmonary congestion in a patient by measuring the patient's thoracic fluid impedance. For example, an implantable cardiac device such as an ICD, CRT-D, or pacemaker can be configured to pass an electrical current across a patient's lung, and to measure the resulting intra-thoracic impedance. As the patient's thoracic fluid accumulates during pulmonary congestion, conductance across the patient's lung increases, causing a corresponding decrease in impedance indicative of the level of thoracic fluid accumulation. Such implantable cardiac devices can also be interrogated by hospital clinicians, allowing the hospital clinicians to monitor the patient's fluid status and to receive early warnings of changes that may signal an impending fluid overload. Based on the patient's monitored fluid status, the hospital clinicians may then determine whether or not it would be appropriate to readmit the patient to the hospital for further monitoring and/or treatment.

### SUMMARY OF THE DISCLOSURE

Systems, methods, and devices for non-invasively detecting and monitoring medical conditions are disclosed. The medical condition may be one of Chronic Obstructive Pulmonary Disease (COPD). According to one implementation, a device for non-invasively detecting and monitoring medical conditions using multiple modalities of sensing, comprises at least two electrodes configured to be positioned on a subject, an acoustic sensor configured to be positioned on a subject, a thoracic impedance measurement module connected to the at least two electrodes, for measuring a first impedance between the at least two electrodes, and a heart acoustic measurement module connected to the acoustic sensor, for detecting and measuring a heart sound from the acoustic sensor. In some implementations, the heart sound is an S3 heart sound. In some implementations, the heart sound is an S4 heart sound. In various implementations, the acoustic sensor is at least one of an ultrasound sensor and a piezoelectric microphone sensor. In some implementations, the at least two electrodes include two electrode pairs, and each electrode pair includes a force electrode and a sense electrode. The force electrode is configured to apply current to the subject and the sense electrode is configured to sense changes caused by the applied current. The changes may include changes in measured voltage.

In some implementations, the device includes a sensor for determining an orientation of the device. In one implementation, the thoracic impedance measurement module measures the first impedance when the device is in a first orientation, and measures a second impedance between the at least two of the surface sensors when the device is in a second orientation. In one example, a first orientation indicates that the device is approximately horizontal, and the second orientation indicates that the device is approximately vertical. In another example, a first orientation indicates that the device is approximately horizontal, and the second orientation indicates that the device is positioned at an angle of between about 30 degrees and about 90 degrees with respect to the horizontal plane. In another example, a first orientation indicates that the device is approximately horizontal, and the second orientation indicates that the device is positioned at an angle greater than about 30 degrees with respect to the horizontal plane. In one example, the second orientation indicates that the device is in a Fowler's position. In some implementations, the thoracic impedance measurement module automatically measures the first impedance at regular intervals.

In one implementation, the device further comprises an electrocardiogram measurement module, connected to the electrodes, for measuring electrical activity between the electrodes.

According to one implementation, a system for non-invasively detecting and monitoring medical conditions using multiple modalities of sensing, comprises a device positioned on a subject having a plurality of surface sensors and a plurality of sensing modules connected to the plurality of surface sensors, configured to gather multi-modality sensing data, and a data analyzer operative to perform at least one of data analysis, data trending, and data reduction of the multi-modality sensing data. The mutli-modality sensing data includes a first impedance between at least two of the surface sensors, and heart sounds from at least one of the plurality of surface sensors. In various implementations, the surface sensors include at least one of electrodes, heart sounds sensors, ultrasound sensors, and photoplethysmography sensors.

In some implementations, the system further includes a data decision engine configured to combine at least some of the multi-modality sensing data, wherein the combined multi-modality sensing data indicates a medical condition status of the subject. In some implementations, the system further includes a transceiver configured to transmit the combined multi-modality sensing data over at least one wireless communication path to a cloud for further processing.

In some implementations, the device in the system further comprises a sensor for determining an orientation of the device. In some implementations, the device includes a thoracic impedance measurement module configured to measure the first impedance when the device is in a first orientation and a second impedance between the at least two of the surface sensors when the device is in a second orientation. In some implementations, the device in the system further comprises an electrocardiogram measurement module, connected to the plurality of surface sensors, for measuring electrical activity between at least two of the surface sensors.

According to some implementations, a method for non-invasively detecting and monitoring medical conditions using multiple modalities of sensing comprises transmitting a current transcutaneously from a first electrode positioned on a subject, receiving a current transcutaneously at a second electrode positioned on the subject, measuring a voltage between the first and second electrodes, determining a thoracic impedance at least based on the voltage, receiving an acoustic signal from an acoustic sensor, measuring a heart sound from the acoustic sensor, and transmitting thoracic impedance data and heart sound measurements to a data analyzer configured to perform at least one of data analysis, data trending, and data reduction of the thoracic impedance data and heart sound measurements. In some implentations, the method further comprises measuring electrical activity between the first and second electrodes and producing an electrocardiogram.

In some implementations, the method further comprises determining an orientation of the device. In some implementations, the thoracic impedance is determined when the device is in a first orientation and the method further comprises determining a second impedance measurement between the first and second electrodes when the device is in a second orientation.

In accordance with the present application, systems, apparatus, and methods are disclosed for non-invasively detecting and monitoring medical or health conditions such as congestive heart failure (CHF) conditions in human subjects using multiple modalities of sensing, including, but not limited to, thoracic impedance sensing, electrocardiogram (ECG) sensing, breath rate sensing, tidal volume sensing, heart sounds sensing, pulse oximetry sensing, blood pressure (systolic, diastolic) sensing, cardiac output sensing, etc. The disclosed systems, apparatus, and methods can non-invasively gather and at least partially analyze, trend, and/or reduce data from each modality of sensing, and perform data fusions on some or all of the multi-modality sensing data in order to obtain curated data useful in detecting the onset of CHF in a human subject and/or monitor its severity. The disclosed systems, apparatus, and methods can also transmit such multi-modality sensing data (as well as other information pertaining to the onset and/or severity of the human subject's CHF) either directly over a communications network to the “cloud,” or to a smartphone or other communications device, which, in turn, can transmit the multi-modality sensing data and/or other information over the communications network to the cloud. The multi-modality sensing data can also be analyzed, trended, reduced, and/or fused in the cloud to augment or at least partially replace the data analysis, trending, reduction, and/or fusion performed by the disclosed systems, apparatus, and methods. The resulting curated multi-modality sensing data and/or other information may then be remotely downloaded from the cloud by hospital clinicians for monitoring and/or tracking purposes. By non-invasively gathering and analyzing data from multiple modalities of sensing to detect the onset of CHF and/or monitor its severity in human subjects, the disclosed systems, apparatus, and methods can increase the positive detection of potentially problematic CHF conditions while decreasing false positives, which can reduce the number of unnecessary hospital readmissions, shorten hospital stays, and reduce hospital costs.

In certain embodiments, a method of non-invasively detecting and monitoring medical or health conditions such as congestive heart failure (CHF) conditions in human subjects using multiple modalities of sensing includes positioning a non-invasive CHF detection and monitoring device on a human subject such that it makes contact with the human subject's torso and upper chest and neck areas or any other suitable parts or areas of the body, via at least a plurality of surface electrodes and/or one or more sensors such as heart sound sensors, ultrasound sensors, photoplethysmography (PPG) sensors, etc. Once the CHF detection and monitoring device is positioned in contact with the human subject's torso and upper chest and neck areas, a plurality of multi-modality sensing and measurement modules contained in the CHF detection and monitoring device are activated to obtain multi-modality sensing data from the human subject. The multi-modality sensing data can include, but are not limited to, one or more of thoracic impedance sensing data, electrocardiogram (ECG) sensing data, breath rate and tidal volume sensing data, heart rate variability/heart sounds-based sensing data, and pulse oximetry sensing data. The multi-modality sensing data are provided to a data analyzer contained in the CHF detection and monitoring device for at least partially analyzing, trending, and/or reducing the data. Next, the analyzed multi-modality sensing data are at least partially fused or combined by a data fusion/decision engine contained in the CHF detection and monitoring device for subsequent use in making one or more inferences about the CHF status of the human subject. The at least partially fused or combined multi-modality sensing data are then transmitted by a transmitter/receiver contained in the CHF detection and monitoring device over one or more wireless communication paths to the cloud for possible further data analysis, trending, reduction, and/or fusion, as well as subsequent remote downloading by hospital clinicians for monitoring and/or tracking purposes.

In certain further embodiments, an apparatus for non-invasively detecting and monitoring medical or health conditions such as CHF conditions in human subjects using multiple modalities of sensing includes a non-invasive CHF detection and monitoring device configured to be positioned on a human subject, thereby making contact with the human subject's torso and upper chest and neck areas or any other suitable parts or areas of the body, via at least a plurality of surface electrodes and/or one or more sensors, such as heart sound sensors, ultrasound sensors, photoplethysmography (PPG) sensors, etc. The CHF detection and monitoring device includes a plurality of multi-modality sensing and measurement modules, a data analyzer, a data fusion/decision engine, and a transmitter/receiver. The plurality of multi-modality sensing and measurement modules are operative to obtain multi-modality sensing data from the human subject, including, but not limited to, one or more of thoracic impedance sensing data, ECG sensing data, breath rate and tidal volume sensing data, heart rate variability/heart sounds-based sensing data, and pulse oximetry sensing data. The data analyzer is operative to perform at least partial data analysis, data trending, and/or data reduction on the multi-modality sensing data. The data fusion/decision engine is operative to at least partially fuse or combine the analyzed multi-modality sensing data for subsequent use in making one or more inferences about the CHF status of the human subject. The transmitter/receiver is operative to transmit the at least partially fused or combined multi-modality sensing data over one or more wireless communication paths to the cloud for possible further data analysis, trending, reduction, and/or fusion, as well as subsequent remote downloading by hospital clinicians for monitoring and/or tracking purposes.

Other features, functions, and aspects of the present application will be evident from the Detailed Description that follows.

### BRIEF DESCRIPTION OF THE DRAWINGS

The accompanying drawings, which are incorporated in and constitute a part of the present application, illustrate one or more embodiments described herein, and, together with the Detailed Description, explain these embodiments. In the drawings:

FIG. 1 is a diagram illustrating an environment including an exemplary system for non-invasively detecting and monitoring medical or health conditions in a subject using multiple modalities, according to some embodiments of the disclosure;

FIG. 2 is a diagram illustrating exemplary functional components of the system of FIG. 1, according to some embodiments of the disclosure; and

FIGS. 3A-3C are diagrams illustrating a device for detecting and monitoring health conditions in a subject, according to some embodiments of the disclosure;

FIG. 4 is a diagram illustrating another device for detecting and monitoring health conditions in a subject, according to some embodiments of the disclosure;

FIG. 5 is a diagram illustrating another device for detecting and monitoring health conditions in a subject, according to some embodiments of the disclosure;

FIG. 6 is a flow diagram illustrating an exemplary method of detecting the onset of a medical or health condition and/or monitoring its severity, according to some embodiments of the disclosure;

FIG. 7 is a diagram illustrating another device for detecting and monitoring health conditions in a subject, according to some embodiments of the disclosure;

FIGS. 8A-8E are diagrams illustrating various examples of electrode and sensor torso placements for detecting and monitoring health conditions in a subject, according to some embodiments of the disclosure;

FIGS. 9A and 9B are diagrams illustrating an exemplary cardiovascular feedback loop; and

FIG. 10 is a diagram illustrating the medical classifications of heart failure.

### DETAILED DESCRIPTION

Systems, apparatus, and methods are disclosed for non-invasively detecting and monitoring medical or health conditions such as congestive heart failure (CHF) conditions and chronic obstructive pulmonary disease (COPD) conditions in human subjects using multiple modalities of sensing. In particular, a device for non-invasively gathering and analyzing, trending, and/or reducing data from each modality of sensing is disclosed. The device can perform data fusions on some or all of the multi-modality sensing data to obtain curated data useful in detecting the onset of a health condition in a human subject and/or monitor its severity, and transmit such multi-modality sensing data (as well as other information pertaining to the onset and/or severity of the human subject's health condition). The data can be transmitted either directly over a communications network to the “cloud,” or to a smartphone or other communications device. A smartphone or other communication device can analyze the data locally, or the smartphone ore other communication device can transmit the multi-modality sensing data and/or other information over the communications network to the cloud. Data transmitted to the cloud can be remotely analyzed, trended, reduced, and/or fused to augment or at least partially replace the data analysis, trending, reduction, and/or fusion performed by the disclosed systems, apparatus, and methods. In some examples, hospital clinicians can remotely download the resulting curated multi-modality sensing data and/or other information from the cloud for monitoring and/or tracking the health status of the human subject.

The disclosed systems, apparatus, and methods for non-invasively detecting and monitoring CHF conditions in human subjects using multiple modalities of sensing can provide improvements over conventional implantable cardiac devices for managing CHF in human subjects, such as implantable cardioverter defibrillators (ICDs), cardiac resynchronization therapy-defibrillators (CRT-Ds), or pacemakers. For example, such conventional implantable cardiac devices typically include one or more sensors configured to provide a single or limited number of sensing modalities, such as a modality for detecting a human subject's fluid retention. However, monitoring and/or tracking the CHF status of a human subject based on just a single or limited number of sensing modalities can often lead to false positives, resulting in unnecessary hospital readmissions that can increase hospital costs. Further, such conventional implantable cardiac devices are generally incapable of analyzing the interrelationship of multi-modality sensing data to obtain positive detection of potentially problematic CHF conditions in human subjects. Moreover, the implantable nature of such conventional cardiac devices can increase surgical risks, as well as the incidence of infection. Additionally, the implantable nature of such conventional cardiac devices limits the availability of the devices to patients, since only patients qualified for the surgery to insert the implant can receive the device.

The disclosed systems, apparatus, and methods for non-invasively detecting and monitoring medical or health conditions such as CHF and COPD conditions in human subjects can non-invasively gather data, and at least partially analyze, trend, and/or reduce data from multiple modalities of sensing. Additionally, the systems, apparatus, and methods can perform data fusions on some or all of the multi-modality sensing data and obtain curated data useful in detecting the onset of CHF and/or COPD, and also useful in monitoring the severity of CHF and/or COPD in human subjects. The disclosed systems, apparatus, and methods thereby increase the positive detection of potentially problematic CHF and COPD conditions while decreasing false positives, which can reduce the number of unnecessary hospital readmissions, shorten hospital stays, and reduce hospital costs. Moreover, the disclosed systems, apparatus, and methods for non-invasively detecting and monitoring CHF and COPD conditions can be implemented in an external device that can be conveniently employed by a human subject following discharge from the hospital, allowing the human subject as well as hospital clinicians to monitor the subject's CHF and/or COPD status with reduced risks from surgery and/or infection.

Worsening heart failure is correlated with changes over time in multiple measurements that can be gathered using the non-invasive systems, apparatus, and methods disclosed herein. In particular, worsening heart failure is correlated with an in increase in amplitude of the S3 heart sound, increasingly rapid and shallow breathing at rest, a decrease in the relative tidal volume (the lung volume representing the volume of air displaced between inhalation and exhalation at rest), and a decrease in thoracic impedance.

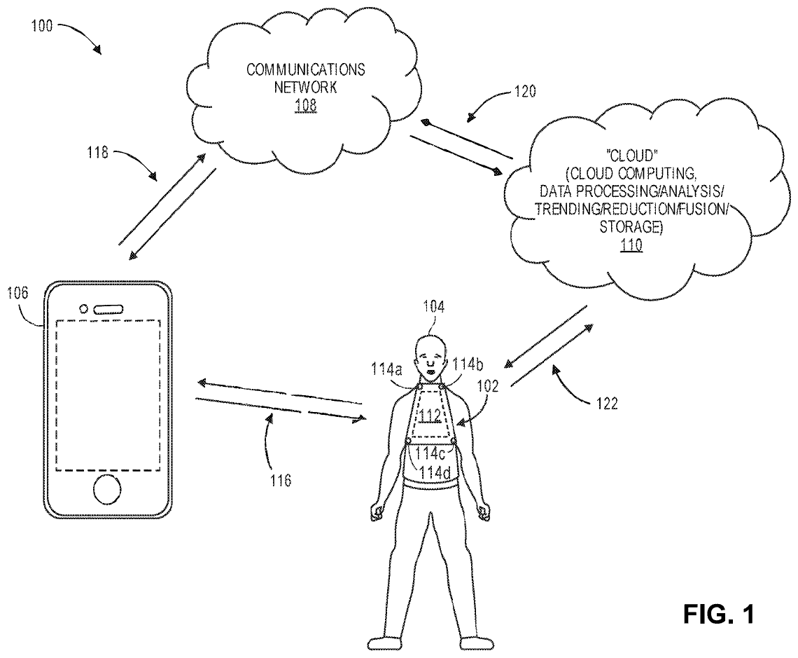

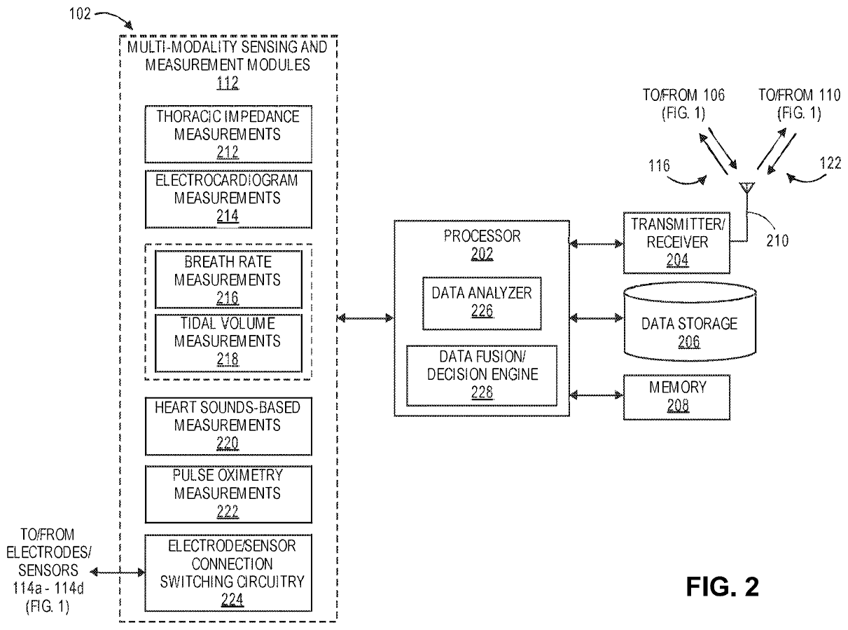

FIG. 1 depicts a typical environment 100 in which an illustrative embodiment of an exemplary system 102 for non-invasively detecting and monitoring medical or health conditions such as congestive heart failure (CHF) conditions in human subjects using multiple modalities of sensing may be employed, according to some embodiments of the disclosure. As shown in FIG. 1, the system 102 includes a plurality of multi-modality sensing and measurement modules 112 (see also FIG. 2), and a plurality of surface electrodes/sensors 114a-114d (e.g., four (4) surface electrodes/sensors, or any other suitable number of surface electrodes/sensors). For example, one or more of the surface electrodes can be implemented as solid-gel surface electrodes, or any other suitable surface electrodes. Further, one or more of the sensors can be implemented as heart sound sensors, ultrasound sensors, photoplethysmography (PPG) sensors, or any other suitable sensors. The system 102 can be configured as a generally triangular-shaped device, or any other suitably shaped device, operative to make contact with one or more of the torso, upper chest, and neck areas, or any other suitable parts or areas of the body, of a human subject 104 via at least the plurality of surface electrodes/sensors 114a-114d.

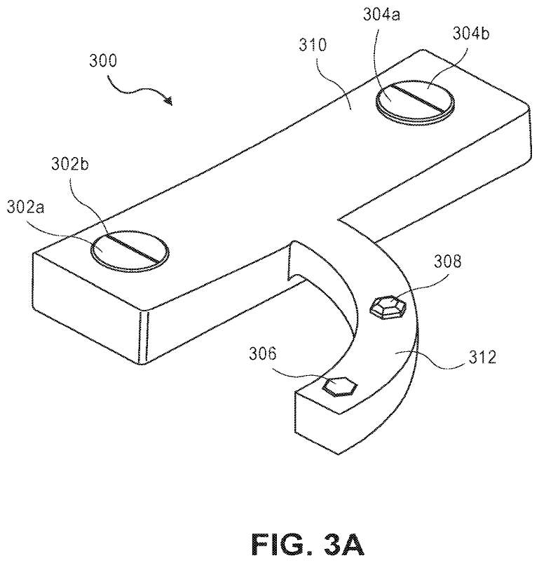

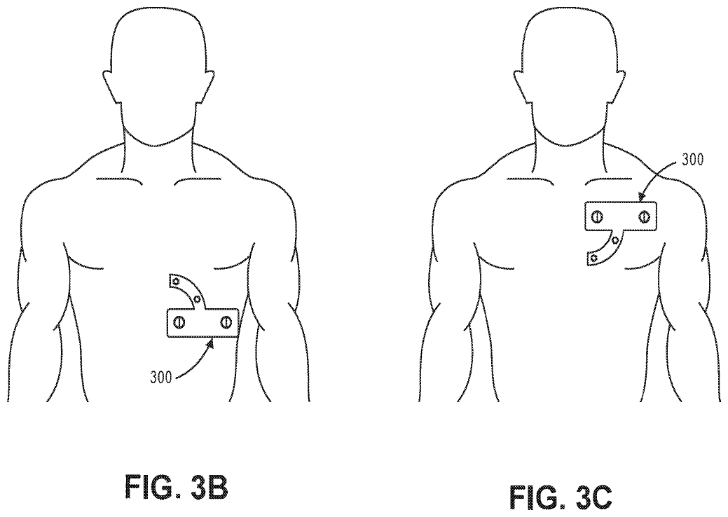

In various implementations, the system 102 can have a configuration that allows it to be implemented within a wearable vest-like structure, as multiple patch-like devices, or any other suitable structure or device(s). Various examples of device configurations are shown in FIGS. 3-5 and FIG. 7.

In the typical environment 100, the system 102 is operative to engage in bidirectional communications over wireless communication paths 116 with a smartphone 106, which, in turn, is operative to engage in bidirectional communications over wireless communication paths 118 with a communications network 108 (e.g., the Internet). The smartphone 106 is further operative, via the communications network 108, to engage in bidirectional communications over wireless communication paths 120 with the cloud 110, which can include resources for cloud computing, data processing, data analysis, data trending, data reduction, data fusion, data storage, and other functions. The system 102 is further operative to engage in bidirectional communications over wireless communication paths 122 directly with the cloud 110.

FIG. 2 depicts a detailed view of the system 102 for non-invasively detecting and monitoring medical or health conditions such as CHF and/or COPD conditions in human subjects, according to some embodiments of the disclosure. As shown in FIG. 2, the system 102 includes the plurality of multi-modality sensing and measurement modules 112, a processor 202 and its associated memory 208, a data storage 206 for storing multi-modality sensing data, and a transmitter/receiver 204. The transmitter/receiver 204 can be configured to perform Bluetooth communications, WiFi communications, or any other suitable short-range communications for communicating with the smartphone 106 (see FIG. 1) over the wireless communication paths 116. The transmitter/receiver 204 can be further configured to perform cellular communications or any other suitable long-range communications for communicating with the cloud 110 (see FIG. 1) over the wireless communication paths 122.

In some implementations, the plurality of multi-modality sensing and measurement modules 112 can include, but are not limited to, one or more of a thoracic impedance measurement module 212, an electrocardiogram (ECG) measurement module 214, breath rate and tidal volume measurement modules 216, 218, a heart sounds-based measurement module 220, and a pulse oximetry measurement module 222. In one embodiment, the system 102 can be configured to perform reflective pulse oximetry measurements. In another embodiment, the system 102 can include a finger-pocket device (not shown) for performing finger-based pulse oximetry measurements. The plurality of multi-modality sensing and measurement modules 112 further include electrode/sensor connection switching circuitry 224 for switchably making connections with the plurality of surface electrodes/sensors 114a-114d shown in FIG. 1.

The processor 202 can include a plurality of processing modules such as a data analyzer 226 and a data fusion/decision engine 228. The transmitter/receiver 204 can include at least one antenna 210 operative to transmit/receive wireless signals such as Bluetooth or WiFi signals over the wireless communications paths 116 to/from the smartphone 106, which can be a Bluetooth or WiFi-enabled smartphone or any other suitable smartphone. The antenna 210 is further operative to transmit/receive wireless signals such as cellular signals over the wireless communications paths 122 to/from the cloud 110.

The operation of the system 102 for non-invasively detecting and monitoring medical or health conditions such as CHF and COPD conditions in human subjects using multiple modalities of sensing will be further understood with reference to the following illustrative example, as well as FIGS. 1 and 2. In this illustrative example, at fixed times each day for a predetermined number of days (e.g., twice a day) while the human subject 104 is in a supine or upright position, the human subject 104 (see FIG. 1) or a human assistant positions the system 102 configured as the generally triangular-shaped device (or any other suitably shaped device) such that it makes contact with one or more of the subject's torso and upper chest and neck areas (or any other suitable parts or areas of the body) via the plurality of surface electrodes/sensors 114a-114d.

Having positioned the system 102 in contact with the human subject's torso and/or upper chest and/or neck areas, the plurality of multi-modality sensing and measurement modules 112 can be activated to gather, collect, sense, measure, or otherwise obtain multi-modality sensing data from the human subject 104. For example, the thoracic impedance measurement module 212 can perform thoracic impedance sensing using multiple vectors to obtain a measure of the human subject's thoracic fluid impedance, as well as trends for obtaining a localization of fluid congestion in the lungs. To that end, the thoracic impedance measurement module 212 can apply, via the electrode/sensor connection switching circuitry 224, a suitable high frequency, low amplitude current between two or more of the surface electrodes 114a-114d. In one example, the current is applied between two electrodes 114a-114d at the neck and thorax of the human subject, such as the surface electrode pair 114a, 114b. The thoracic impedance measurement module 212 obtains, via the electrode/sensor connection switching circuitry 224, a thoracic impedance signal by measuring the potential difference between two of the surface electrodes 114a-114d. In some examples, the high frequency, low amplitude current applied between the surface electrodes 114a-114d has a frequency between about 50 kHz and about 100 kHz and has an amplitude between about 1 mAᵣₘₛ and 4 mAᵣₘₛ. In other examples, the current has a frequency below about 50 kHz or above about 100 kHz. In some examples, the current has a frequency of between about 20 kHz and about 200 kHz, or between about 20 kHz and about 1 MHZ.

In some implementations, the thoracic impedance measurement module 212 uses measurements obtained from two pairs of surface electrodes. In one implementation, four electrodes are used for impedance measurements. The four electrodes include two force electrodes and two sense electrodes, with each force electrode paired with a sense electrode. Each group of four electrodes can resolve a vector in space to localize observed changes. There are two of the four electrodes on each side of a vector. In particular, on each side of a vector, there is a force electrode and a sense electrode. The force electrode applies (or injects) current into the body (or receives current injected into the body). The sense electrode measures the disturbance caused by the current applied into the body by the force electrode. In various implementations, the sense electrodes of the vector sense current and/or voltage drop caused by the injection of a current into the body by the force electrodes through application of a voltage and/or current. Since voltage and current are related to impedance (V=Z*i), to measure impedance Z, a known current i can be applied, and the subsequent voltage drop V can be measured, and Z can be calculated using the known current i and measured change in voltage V. According to various implementations, the properties of the circuitry for applying current (the force electrode) are different from the properties of the circuitry for measuring the voltage (the sense electrodes). According to various implementations, there are two different sets of electrodes (force electrodes and sense electrodes), and each electrode has a positive and a negative side.

Using the four electrodes, different parts of the tissue, and tissue at varying depths can be scanned by adjusting the frequency of the injected waveform. Impedance is measured on a single vector without spatial resolution. In some implementations, more than four electrodes are used, and additional electrode pairs add multiple vectors, which add additional tissue scans. In some implementations, each pair of sense and force electrodes is one side of multiple vectors. For example, two pairs of sense and force electrodes form one vector, and three pairs of sense and force electrodes form three vectors. In other examples, more pairs of electrodes are used and more vectors are formed. As such, each vector monitors a selected spatial area.

The measurement of impedance can be used to determine physiologic information, including respiration rate, tidal volume, and lung fluids. Impedance measurements can also be used to determine derived metrics such as pulmonary resistance and lung fluid location. For respiration rate, the respiration of a patient causes air to go into the lungs and increase the lung volume which compresses the surrounding tissue. This leads to changes in the impedance with an increase on some vectors (mainly vectors that cross the lung(s)) and a decrease in other vectors other due to redirection of the current. Respiration rate is determined based on the changes in impedance, which follow the same periodicity as the respiration. Additionally, tidal volume can be monitored by determining the amplitude of the changes, which are proportional to the tidal volume (change in lung volume). The shape of the waveform relates to the breathing pattern and can be used to monitor the airway/lung resistance.

Fluid in the lungs can be detected and/or monitored by scanning multiple frequencies and/or multiple spatial vectors, which are used to distinguish fluids in the lung vs. other bodily fluids. Another method to add to the specificity of the separation between lung fluid and other bodily fluids is to measure impedance changes with posture. Changes in posture can cause lung fluids to move with gravity. The movement in fluids is detected by various vector measurements and helps separate the moving lung fluids from other bodily fluids. In one example, the vector of impedance is measured at the bottom of the lungs using a single frequency of 50 KHz. For example, the impedance vector between electrodes 114c and 114d of FIG. 1 may be measured. If the impedance vector is measured when the person is supine (horizontal position) and then is measured again after the patient moves to the standard Fowler position, a change in the impedance of 1 ohm or more can indicate the presence of fluid in the lungs. In congestive heart failure patients, the change in impedance is typically more than 5 ohms when patients start experiencing symptoms.

Note that in medicine, Fowler's position is a standard patient position in which the patient is seated in a semi-upright sitting position with the patient's torso at an angle relative to the horizontal plane. In various examples, Fowler's position includes the patient's torso being at an angle of between about 15 and thirty degrees, the patient's torso being at an angle of between about 30 and about 45 degrees, the patient's torso being at an angle of between about 45 and about 60 degrees, and the patient's torso being an angle of between about 60 and about 90 degrees. In standard Fowler's position, the patient's torso is between about 45 and about 60 degrees.

In various implementations, the device includes a sensor that can determine the position of the device, thereby indicating the horizontal position of the patient, including whether the patient is upright or supine. The device automatically measures thoracic impedance in both upright and supine positions of the patient, and uses these two measurements to monitor and/or detect CHF.

As discussed above, the device also includes an electrocardiogram (ECG) measurement module 214, which can perform ECG measurements at some or all of the plurality of surface electrodes 114a-114d that make contact with the skin of the human subject 104 on his or her torso, upper chest, and/or neck areas. In one embodiment, the pulse oximetry measurement module 222 (or the finger-pocket device for performing finger-based pulse oximetry measurements) can be employed in conjunction with the ECG measurement module 214 to obtain further measurements. Because respiratory activity can cause corresponding changes in the measured thoracic impedance, each of the breath rate measurement module 216 and the tidal volume measurement module 218 can operate in conjunction with the thoracic impedance measurement module 212 to obtain measurements of the human subject's breath rate/breath rate variability and tidal volume, respectively.

The heart sounds-based measurement module 220 can include an electronic stethoscope, stethophone, or any other suitable device for obtaining heart rate variability data, as well as obtaining and converting heart sounds (e.g., the S1 heart sound, “lub”; the S2 heart sound, “dub”) to sensing data that can be subsequently algorithmically analyzed by the data analyzer 226 to obtain information pertaining to the S3 heart sound (also known as the proto-diastolic or ventricular gallop) which may be heard at the beginning of diastole, during the rapid filling of the ventricles, and the S4 heart sound (also known as the atrial gallop) which may heard late in diastole. In one implementation, the heart sounds-based measurement module 220 measures sub-audible heart sounds (heart sounds below about 40 Hz), thereby measuring S3 and S4 heart sounds that cannot be heard by a physician or other health care professional using a stethoscope. S3 and S4 heart sounds are pathological and indicate heart failure. Information about sub-audible S3 and S4 heart sounds can be used to detect and monitor CHF. The heart sounds can be measured by a sensor placed over the heart region. The sensor detects sounds and/or vibrations. The sensor detects different heart sounds depending on the location of sensor placement relative to the various regions of the heart. For example, to maximize the chances of detecting the signal that is pertinent to S3, the sensor is positioned over the apex of the heart, which is in the fifth intercostal space. Abnormal S3 heart sounds occur when the heart pumping is compromised. The S3 heart sounds are early indicators of heart problems and can change prior to other measureable heart signals. Because the S3 heart sounds have a lot of energy at low frequencies that are not audible to the human car, the S3 heart sounds are initially undetectable by physicians. A sensing system sensitive to low frequencies coupled with an automated algorithm can detect the S3 heart sounds earlier. Any presence of S3 or S4 heart sounds in adult patients is abnormal and the detection of any energy that is determined to be S3 or S4 can be used to flag a potential problem. The flags from different sensors can be combined by a higher level logic to generate a single metric that can be designed to be more specific and more sensitive than individual measures.

In one embodiment, the pulse oximetry measurement module 222 (or the finger-pocket device for performing finger-based pulse oximetry measurements) can be employed in conjunction with the heart sounds-based measurement module 220 to obtain further measurements. It is noted that the pulse oximetry measurement module 222 can perform reflective or finger-based pulse oximetry measurements. In one embodiment, the pulse oximetry measurement module 222 can include a pulse rate sensor, as well as a blood oxygen level (SpO₂) sensor.

Having performed the thoracic impedance measurements, the ECG measurements, the breath rate and tidal volume measurements, the heart rate variability/heart sounds-based measurements, and the pulse oximetry measurements, the thoracic impedance measurement module 212, the electrocardiogram (ECG) measurement module 214, the breath rate and tidal volume measurement modules 216, 218, the heart sounds-based measurement module 220, and the pulse oximetry measurement module 222 provide corresponding multi-modality sensing data to the data analyzer 226 for at least partial data analysis, data trending, and/or data reduction. In one embodiment, such multi-modality sensing data can also be analyzed, trended, and/or reduced “in the cloud” and made available in cloud-based data storage 110 with pre-set alerts for use in various levels of clinical interventions. For example, the data analyzer 226 can (1) analyze the thoracic impedance measurement data to obtain information pertaining to the human subject's lung congestion, (2) analyze the breath rate and tidal volume measurement data to obtain information pertaining to the human subject's shortness of breath (e.g., dyspnea, paroxysmal nocturnal dyspnea), (3) analyze the ECG measurement data and heart rate variability data in multiple (e.g., 3) projections to obtain information pertaining to possible atrial fibrillation and localization in the human subject 104, and (4) analyze the heart sounds-based measurement data to obtain information pertaining to a possible increase in the S3 heart sound (which can be indicative of a failing left ventricle due to a dilated CHF condition).

The data analyzer 226 provides the at least partially analyzed multi-modality sensing data to the data fusion/decision engine 228, which effectively at least partially fuses or combines the multi-modality sensing data, in accordance with one or more algorithms and/or decision criteria, for subsequent use in making one or more inferences about the CHF status of the human subject 104. For example, combined multi-modality sensing data that show, substantially concurrently, an increase in the S3 heart sound, an increase in rapid shallow breathing while the human subject 104 is at rest, a decrease in the relative tidal volume, and a decrease in the thoracic impedance, can be a strong predictor of a potentially problematic CHF condition in the human subject 104. In one embodiment, such algorithms and/or decision criteria implemented in the data fusion/decision engine 228 can be proven and/or refined through one or more clinical trials for strengthening the inferences made by the data fusion/decision engine 228 regarding the human subject's CHF status. The processor 202 then provides the at least partially combined multi-modality sensing data to the transmitter/receiver 204, which transmits the combined multi-modality sensing data either directly over the wireless communication paths 122 to the cloud 110, or over the wireless communication paths 116 to the smartphone 106. Next, the smartphone 106 can transmit, via the communications network 108, the combined multi-modality sensing data over the wireless communication paths 118, 120 to the cloud 110, where it can be further analyzed, trended, reduced, and/or fused. The resulting curated multi-modality sensing data can then be remotely downloaded by hospital clinicians for monitoring and/or tracking purposes.

FIG. 3A is a diagram illustrating a device 300 for detecting and monitoring health conditions in a subject, according to some embodiments of the disclosure. In particular, the device 300 non-invasively detects and monitors CHF conditions in human subjects. The device 300 includes a first electrode pair 302a-302b, a second electrode pair 304a-304b, a third electrode 306, and a fourth electrode 308. The first electrode pair 302a-302b includes one force electrode and one sense electrode, as described above. Similarly, the second electrode pair 304a-304b includes one force electrode and one sense electrode. The third 306 and fourth 308 electrode can be any type of electrode, including, for example, one of a heart sound sensor, a force electrode and, a sense electrode. The device 300 has an elongated rectangular base portion 310, which includes the first electrode pair 302a-302b positioned on a first end and the second electrode pair 304a-304b positioned on a second end. In various examples, the elongated base portion 310 is between about 10 cm and about 20 cm, and between about 2 cm and about 6 cm wide. In some examples, the elongated base portion 310 is between about 1 cm and about 4 cm thick. Along the mid-section of the length of the rectangular base portion 310, a curved tail portion 312 extends out in the same plane as the rectangular base portion 310 and arcs around toward the direction of the first electrode pair 302a-302b. The curved tail portion 312 includes the third electrode 306 and the fourth electrode 308. The third electrode 306 is positioned at the end of the curved tail portion 312 and the fourth electrode 308 is positioned in the middle of the curved tail portion 312. According to various implementations, the device 300 is placed on a subject's torso, with the electrodes 302a-302b, 304a-304b, 306, 308 in contact with the subject's skin.

FIG. 3B shows the device 300 positioned on the torso of a subject. The elongated base portion 310 is positioned around the sixth intercostal space, and the curved tail portion 312 extends upwards, curving inward toward the midline of the torso. In some implementations, the fourth electrode 308 is positioned in the fifth intercostal space on the left side of the torso, and is optimally positioned to detect S3 sounds. In other implementations, the curved tail portion 312 curves outward away from the midline of the torso.

FIG. 3C shows the device 300 positioned on the torso of a subject. The elongated base portion 310 is positioned below the shoulder level, and the curved tail portion 312 extends downwards over the heart, curving inward toward the midline of the torso. In other implementations, the curved tail portion 312 curves outward away from the midline of the torso.

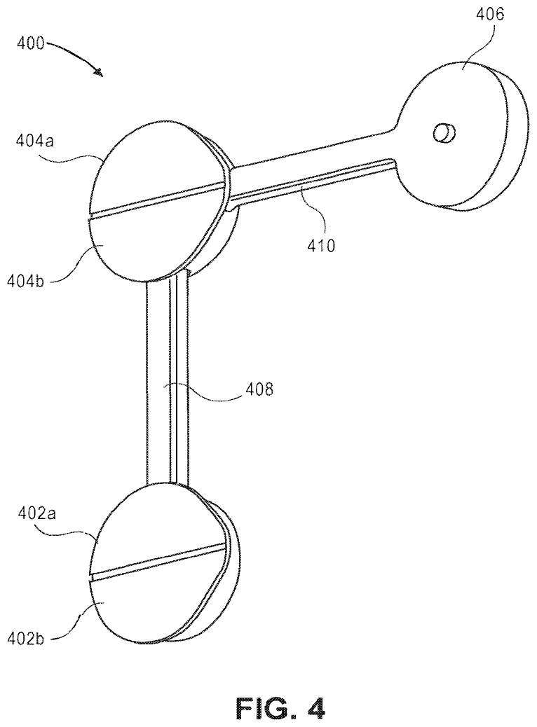

FIG. 4 is a diagram illustrating a device 400 for detecting and monitoring health conditions in a subject, according to some embodiments of the disclosure. In particular, the device 400 non-invasively detects and monitors CHF conditions in human subjects. The device 400 includes a first electrode pair 402a-402b, a second electrode pair 404a-404b, and a sensor 406. The first electrode pair 402a-402b includes a force electrode and a sense electrode. Similarly, the second electrode pair 404a-404b includes a force electrode and a sense electrode. Additionally, one of the first electrode pair 402a-402b and one of the second electrode pair 404a-404b measures ECG. The sensor 406 is a heart sounds sensor for detecting sound vibrations. In one implementation, the sensor 406 is a piezo electric microphone. The membrane of the microphone protrudes to contact the torso and detect the heart sounds. The first electrode pair 402a-402b is connected to the second electrode pair 404a-404b via a first elongated element 408. The second electrode pair 404a-404b is connected to the sensor 406 via a second elongated element 410. As shown in FIG. 4, in the device 400, the first 408 and second 410 elongated elements are approximatly perpendicular to each other. In other implementations, the first 408 and second 410 elongated elements can be oriented at any selected position relative to each other. The device 400 is placed on a subject's torso, with the electrodes 402a-402b and 404a-404b, and the sensor 406 in contact with the subject's skin. In some implementations, the electrodes 402a-402b and 404a-404b, are positioned along the sixth intercostal space and the sensor 406 is positioned along the fifth intercostal space at the apex of the heart.

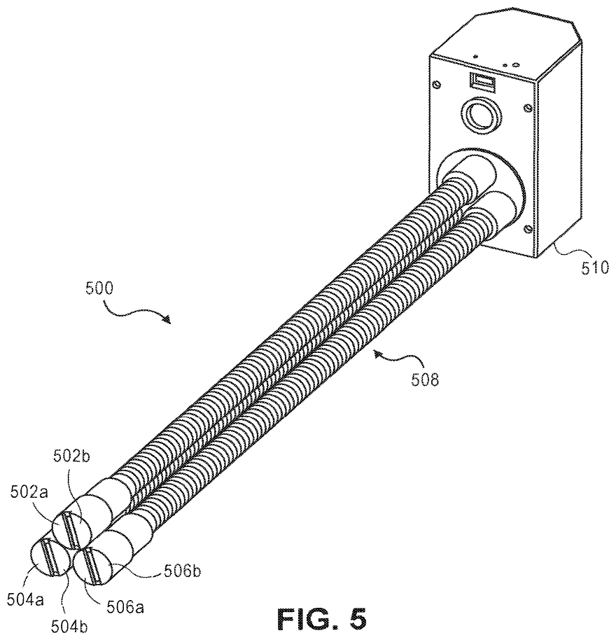

FIG. 5 is a diagram illustrating a device 500 for detecting and monitoring health conditions in a subject, according to some embodiments of the disclosure. The device 500 includes three long flexible arms 508 that extend from a rectangular box 510. At the tip of each of the flexible arms 508 is a pair electrodes-first electrode pair 502a-502b, second electrode pair 504a-504b, and third electrode pair 506a-506b. The electrode pair 502a-502b, 504a-504b, 506a-506b can be moved to be oriented to fit a subject's body and secured in place. The device 500 is a handheld device that the subject places on the body. In some examples, the subject positions the device 500 on the body regularly, such as two or more times per day, and records measurements. According to some implementations, the device 500 includes a sensor for recording heart sounds. The heart sound sensor may also be attached to the device via a long flexible arm. The heart sound sensor may be a microphone, and in some examples it is a piezo electric microphone.

In other implementations, electrodes and/or other sensors are positioned subcutaneously on a subject. Subcutaneous sensors can remain in place long term and can be connected to an external device for measurements. In other implementations, electrodes and/or other sensors are positioned within patches which attach to a subject's skin. The patches are connected to an external device for measurement.

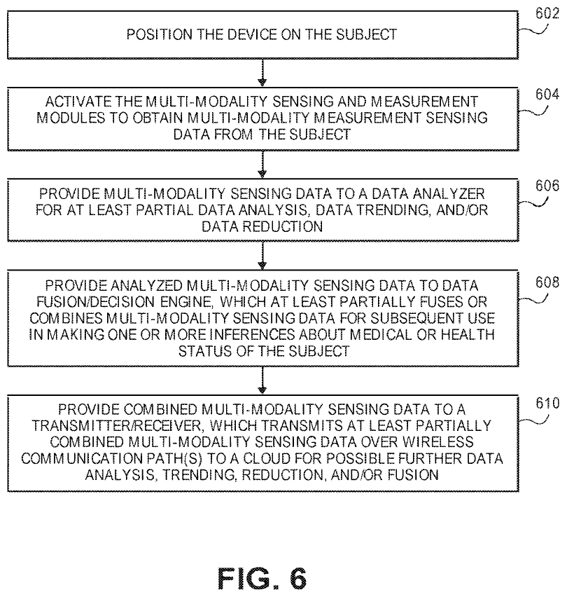

An exemplary method of non-invasively detecting and monitoring medical or health conditions in human subjects using multiple modalities of sensing is described below with reference to FIG. 6, as well as FIGS. 1 and 2. At block 602, the system 102 (see FIGS. 1 and 2), configured as a suitably shaped device, is positioned on the human subject 104 such that it makes contact with suitable parts or areas of the body via at least the plurality of surface electrodes/sensors 114a-114d. At block 604, once the system 102 is positioned in contact with the suitable parts or areas of the body, the plurality of multi-modality sensing and measurement modules 112 are activated to obtain multi-modality sensing data from the human subject 104, including, but not limited to, one or more of thoracic impedance sensing data, ECG sensing data, breath rate and tidal volume sensing data, heart rate variability/heart sounds-based sensing data, and pulse oximetry sensing data. At block 306, the multi-modality sensing data are provided to the data analyzer 226 for at least partial data analysis, data trending, and/or data reduction. At block 608, the analyzed multi-modality sensing data are provided to the data fusion/decision engine 228, which effectively at least partially fuses or combines the multi-modality sensing data for subsequent use in making one or more inferences about the medical or health status of the human subject 104. At block 610, the combined multi-modality sensing data are provided to the transmitter/receiver 204, which transmits the at least partially combined multi-modality sensing data over the wireless communication paths 122 to the cloud 110 for possible further data analysis, trending, reduction, and/or fusion. The partially combined multi-modality sensing data can also be transmitted over the wireless communication paths 122 to the cloud 110 for remote downloading by hospital clinicians for monitoring and/or tracking purposes.

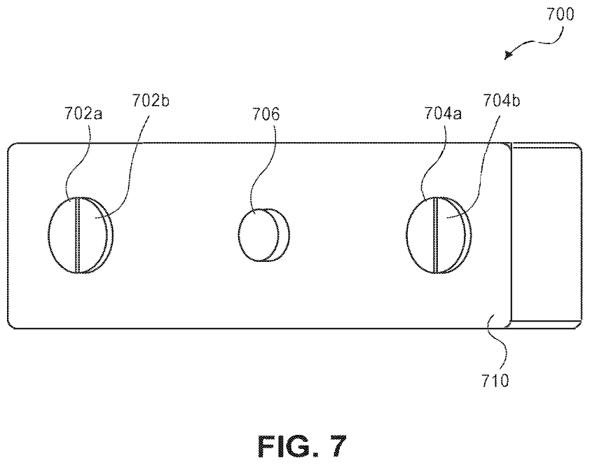

FIG. 7 is a diagram illustrating a device 700 for detecting and monitoring health conditions in a subject, according to some embodiments of the disclosure. In particular, the device 700 non-invasively detects and monitors COPD conditions in human subjects. The device 700 is an elongated rectangular clement 704 and includes first 702a, second 702b, and third 702c electrodes. The first electrode 702a is positioned at a first end of the elongated rectangular clement 704, the second electrode 702b is positioned approximately in the center of the elongated rectangular element 704, and the third electrode 702c is positioned at a second end of the elongated rectangular element 704. In use, the device 700 is positioned on the torso of a subject with the electrodes 702a-702c positioned in contact with the subject's skin. As described above with respect to the system 102 of FIG. 1, data from the electrodes 702a-702c are connected to a plurality of multi-modality sensing and measurement modules (such as the modules 112 shown in FIG. 2), which can be activated to gather, collect, sense, measure, or otherwise obtain multi-modality sensing data from the subject.

To detect and/or monitor for COPD, the heart sounds sensor is positioned higher on the torso for better detection of lung sounds. Measurements for COPD include impedance for determining respiration rate, ECG if desired, impedance for determining tidal volume, lung sounds (for detection of abnormal lung rails), and impedance for measuring the shape of the lung volume changes. In particular, the change in the shape of the impedance variations indicates the lung/airway resistance, which can be used to determine the presence of COPD.

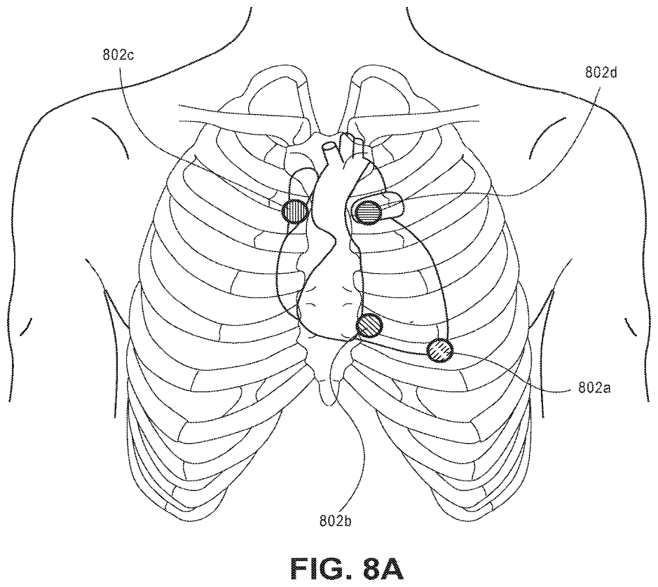

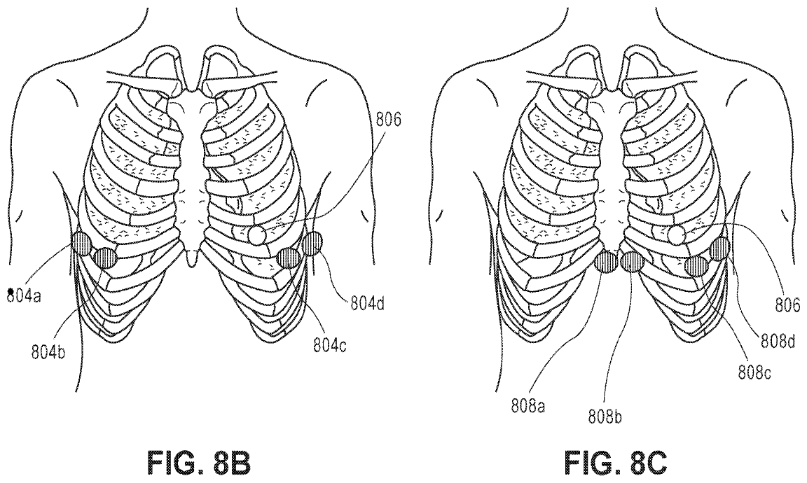

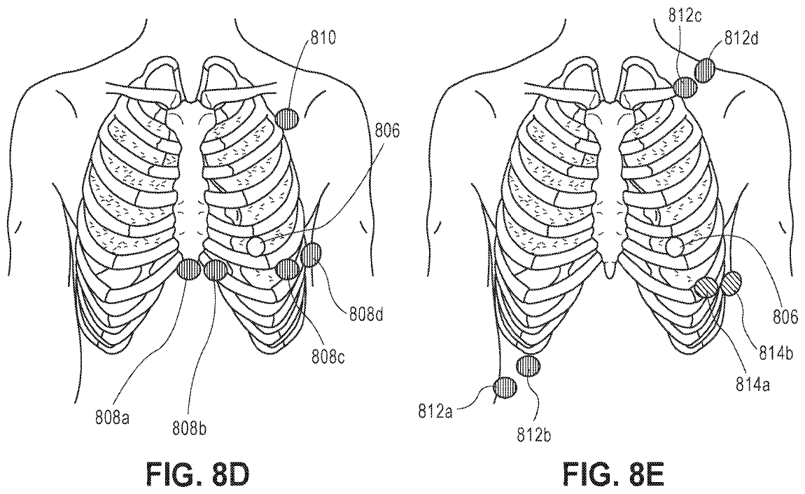

FIGS. 8A-8E are diagrams illustrating various examples of electrode and sensor torso placements for detecting and monitoring health conditions in a subject, according to some embodiments of the disclosure. In FIG. 8A, four elements 802a-802d are positioned on the torso of the subject, generally over the subject's heart. One element 802a is positioned at the apex of the heart, in the fifth intercostal space. According to some examples, the placement of 802a at the apex of the heart in the fifth intercostal space is generally optimal for detection of S3 and S4 heart sounds.

In FIG. 8B, elements 804a-804d are positioned across both sides of the torso, and are thereby positioned to measure the impedance across both lungs. The element 806 is a microphone for detecting heart sounds. A device including elements 804a-804dextends across the width of the torso. In some implementations it may be secured in place with a strap around the torso. The elements 804a-804d can also be used to measure an ECG.

In FIG. 8C, elements 808a-808d are positioned on one side of the torso and measure the impedance across one lung. The element 806 is a microphone for detecting heart sounds. A device including elements 808a-808d extends across one side of the torse. In one example, a device such as the device 300 in FIG. 3A can include elements 808a-808d and 806. The elements 808a-808d can also be used to measure an ECG.

FIG. 8D shows the elements 808a-808d and 806 of FIG. 8C, and an additional clement 810. The element 810 can be used with any of the elements 808a-808d to measure an ECG.

FIG. 8E shows elements 812a-812d, 814a-814b, and 806. Elements 812a-812d and 814a-814b can be used to measure impedance and to measure an ECG. The configuration of elements 812a-812d, 814a-814b and 806 can be implemented in a device such as a vest or a shirt.

Having described the above illustrative embodiments of systems, apparatus, and methods of non-invasively detecting and monitoring medical or health conditions such as CHF conditions in human subjects using multiple modalities of sensing, other alternative embodiments and/or variations can be made and/or practiced. For example, it was described herein that the thoracic impedance measurement module 212, the ECG measurement module 214, the breath rate and tidal volume measurement modules 216, 218, the heart sounds-based measurement module 220, and the pulse oximetry measurement module 222 can provide corresponding multi-modality sensing data to the data analyzer 226 for subsequent data analysis, data trending, and/or data reduction. In an alternative embodiment, one or more of the plurality of multi-modality sensing and measurement modules 112 can further obtain multi-modality sensing data pertaining to non-invasive, pressure wave velocity (PWV)-based systolic and/or diastolic blood pressures between the human subject's chest and finger, for example, and/or cardiac output data based on the direct measurement of at least the cardiac contractility, and provide these additional modalities of sensing data to the data analyzer 226 for further data analysis, data trending, and/or data reduction.

It will be appreciated by those of ordinary skill in the art that modifications to and variations of the above-described systems, apparatus, and methods may be made without departing from the inventive concepts disclosed herein. Accordingly, the present application should not be viewed as limited except as by the scope and spirit of the appended summary of important aspects.

### APPENDIX

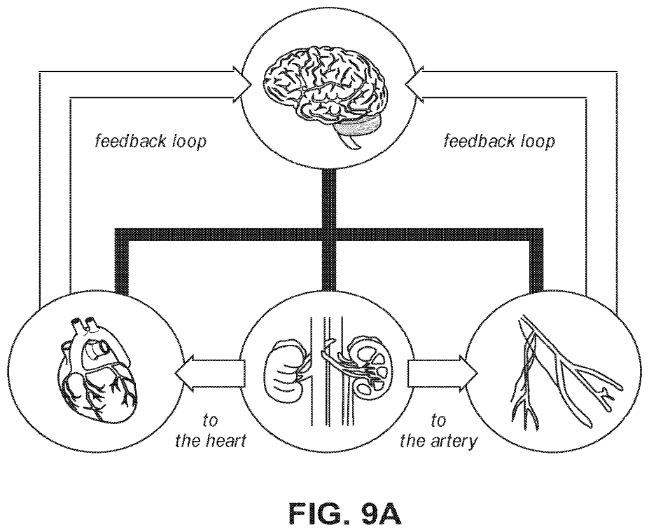

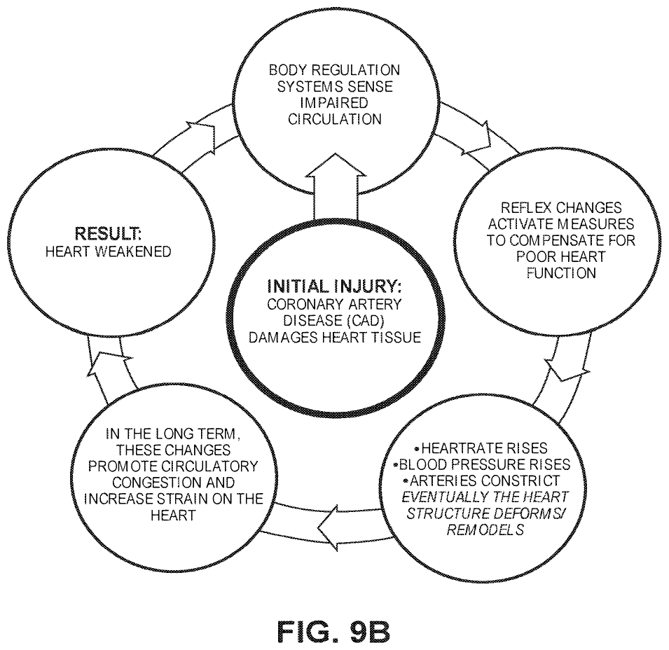

### Cardiovascular Feedback Loop (see FIGS. 9A and 9B)

The nervous system receives a signal that blood pressure is falling. To ensure survival, the brain sends signals to the heart, kidneys, and arteries, each playing a role in diverting blood flow to major organs and maintaining blood pressure. In response to the signals from the brain, the heart beats faster and more forcefully. This increases the circulating blood pressure to the body. It then sends feedback to the brain, informing that changes have been made, and to halt the nervous system's intervention. The signals from the brain stimulate the adrenal glands (a member of the endocrine system located on top of each kidney) to secrete epinephrine (generally known as adrenaline) and norepinephrine into the bloodstream. Upon reaching target organs, it alters their activity. The arteries, in response to the signals from the brain, assist the blood pressure in the body by changing arterial resistance to flow. Changes in vascular tone shift blood away from muscles to internal organs, as their health most directly effects survival. Arteries send feedback to the brain, informing it of the changes.

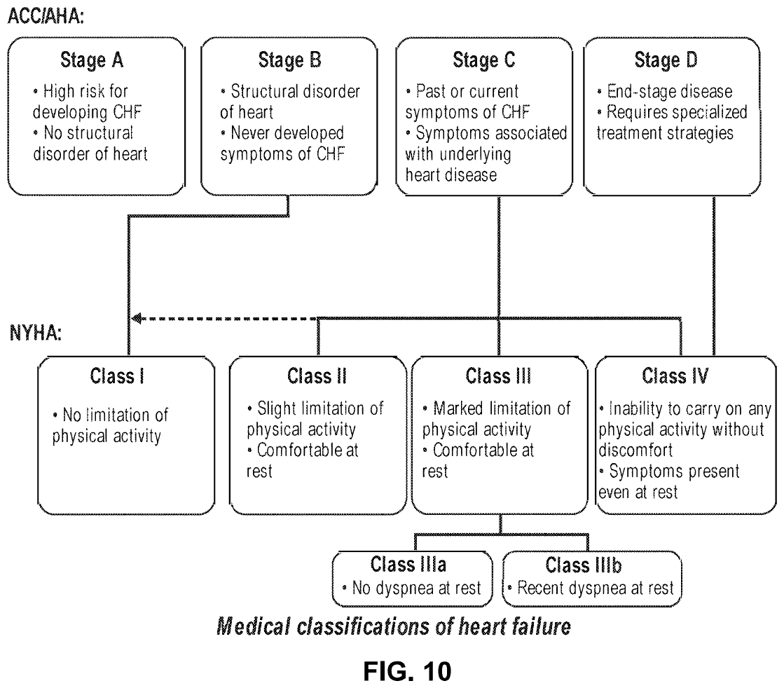

### What is “Heart Failure?” (see FIG. 10)

The 2013 ACCF/AHA Heart Failure Guidelines defined heart failure as “a complex clinical syndrome that results from any structural or functional impairment of ventricular filling or ejection of blood.” Heart pump impairment resulting in symptoms:

Heart: It is necessary that any occurrence of clinical heart failure must include a primary or secondary involvement of the heart. If this is not evident, we either have not looked hard enough or it is not heart failure.

Pump: A complete description of the heart includes multiple functions including electrical, hormonal, and structural components. For heart failure to be present, however, there must be a manifest effect on the ability of the heart to move blood in the circulation.

Impairment: Impairment implies a degree of insufficiency that, in general, does not require complete replacement therapy. It may only be unmasked by activity or stress. If given a letter grade, many patients' heart function would rate a C+rather than an F. Nevertheless, some degree of decreased function must be present for an individual to have heart failure.

Resulting in: An initial insult to the heart may result in an immediate profound or a subtle progression to heart pump impairment only over time. Neurohormonal mechanisms may be activated and contribute to this syndrome. This may include adverse structural and biochemical remodeling. Any process affecting the heart must be causally related to an individual's status to result in heart failure.

Stages: The ACC/AHA classification of heart failure defines 4 stages of heart failure beginning with (1) risk factors for heart failure, (2) asymptomatic heart impairment, (3) symptomatic heart failure, and (4) advanced heart failure.

What is claimed is:

1. A system to detect and monitor medical or health conditions of a subject, comprising:
multiple electrodes configured to be in contact with a torso of the subject;
an acoustic sensor configured to detect at least one of heart sounds or lung sounds from the torso of the subject;
a first measurement module connected to the multiple electrodes, the first measurement module configured to measure a thoracic impedance using a first pair of electrodes of the multiple electrodes;
a second measurement module connected to the acoustic sensor, the second measurement module configured to detect and measure one or more heart sounds via the acoustic sensor;
a third measurement module connected to the multiple electrodes, the third measurement module configured to measure electrical activity between a second pair of electrodes of the multiple electrodes; and
a processor coupled with the first measurement module, the second measurement module, and the third measurement module, wherein the processor is configured to:
perform data trending of at least one of thoracic impedance measurement data, heart sound measurement data, or electrocardiogram measurement data;
identify, based on the data trending, one or more changes over time in the at least one of the thoracic impedance measurement data, the heart sound measurement data, or the electrocardiogram measurement data; and
generate, based on the one or more changes over time, inference information indicating a medical or health status of the subject.

2. The system of claim 1, wherein the first pair of electrodes and the second pair of electrodes have a same two electrodes.

3. The system of claim 1, wherein the first pair of electrodes and the second pair of electrodes have at least one different electrode.

4. The system of claim 1, wherein to measure the thoracic impedance using the first pair of electrodes,
an electrode of the first pair of electrodes is configured as a first sense electrode,
another electrode of the first pair of electrodes is configured as a second sense electrode,
an electrode of a third pair of electrodes of the multiple electrodes is configured as a first force electrode, and
another electrode of the third pair of electrodes is configured as a second force electrode,
wherein the first force electrode is configured to inject current into the torso of the subject and the second force electrode is configured to receive the current injected into the body of the subject, and
wherein the first sense electrode and the second sense electrode are configured to sense a voltage difference caused by the current injected into the torso of the subject,
with the measured thoracic impedance being based on the voltage difference and the current applied to the torso of the subject.

5. The system of claim 1, wherein the processor is located in a cloud computing device.

6. The system of claim 1, further comprising a wearable device containing the processor, the first measurement module, the second measurement module, the third measurement module, the acoustic sensor, and the multiple electrodes.

7. The system of claim 6, wherein the wearable device further contains a transceiver configured to transmit at least a portion of the thoracic impedance measurement data, the heart sound measurement data, and the electrocardiogram measurement data over at least one wireless communication path to a cloud computing device for processing.

8. The system of claim 1, wherein:
the thoracic impedance measurement data comprises measured impedance data and first-determined data based on the measured impedance data;
the heart sound measurement data comprises measured heart sound data and second-determined data based on the measured heart sound data; and
the electrocardiogram measurement data comprises measured electrocardiogram data and third-determined data based on the measured electrocardiogram data.

9. The system of claim 8, wherein:
the first-determined data based on the measured impedance data comprises one or more of respiration rate data, tidal volume data, pulmonary resistance data, or lung fluid data;
the second-determined data based on the measured heart sound data comprises diastolic heart sound data; and
the third-determined data based on the measured electrocardiogram data comprises heart rate variability data.

10. The system of claim 9, wherein the first-determined data based on the measured impedance data comprises the respiration rate data, wherein the respiration rate data is based on periodic changes in measured impedance data having a waveform that corresponds to a breathing pattern, the periodic changes having a periodicity corresponding to periodicity of respiration.

11. The system of claim 9, wherein the first-determined data based on the measured impedance data comprises the tidal volume data, wherein the tidal volume data is determined as proportional to an amplitude of changes in measured impedance data having a waveform that corresponds to a breathing pattern.

12. The system of claim 9, wherein the first-determined data based on the measured impedance data comprises the lung fluid data, wherein the lung fluid data is based on a posture of the subject.

13. The system of claim 12, wherein the lung fluid data includes a first impedance vector measured when the subject is in a horizontal position and a second impedance vector measured when the subject is in a non-horizontal position.

14. The system of claim 1, wherein the acoustic sensor is positioned on the torso of the subject.

15. The system of claim 8, wherein the processor is further configured to combine at least two of the thoracic impedance measurement data, the heart sound measurement data, or the electrocardiogram measurement data, resulting in combined multi-modality sensing data to predict worsening of a cardiopulmonary condition of the subject.

16. The system of claim 15, wherein the processor is configured to:
detect a potentially problematic cardiopulmonary condition based on the changes over time in at least one of the thoracic impedance measurement data, the heart sound measurement data, or the electrocardiogram measurement data.

17. The system of claim 16, wherein the potentially problematic cardiopulmonary condition is associated with worsening heart failure.

18. The system of claim 16, wherein to detect the potentially problematic cardiopulmonary condition the processor is further configured to identify a diastolic heart sound.

19. The system of claim 16, wherein to detect the potentially problematic cardiopulmonary condition the processor is further configured to:
identify an in increase in energy of a diastolic heart sound;
identify an increasingly rapid and shallow breathing rate at rest;
identify a decrease in a relative tidal volume; and
identify a decrease in the thoracic impedance.

20. The system of claim 16, wherein the thoracic impedance measurement data comprises lung fluid data, wherein the lung fluid data includes a first measured impedance data when the subject is in a horizontal position and a second measured impedance data when the subject is in a non-horizontal position; and
wherein to detect the potentially problematic cardiopulmonary condition the processor is further configured to:
identify a change of more than 5 ohms between the first measured impedance data and the second measured impedance data.

21. The system of claim 15, wherein to predict worsening of the cardiopulmonary condition of the subject, the processor is further configured to:
analyze the thoracic impedance measurement data to identify a decrease in the thoracic impedance;
analyze the thoracic impedance measurement data to identify an increase in a respiration rate;
analyze the thoracic impedance measurement data to identify a decrease in a relative tidal volume;
analyze the electrocardiogram measurement data and heart rate variability data to obtain information pertaining to possible atrial fibrillation and localization in the subject; and
analyze the heart sound measurement data to identify an increase in an S3 heart sound.

22. The system of claim 1, wherein the first measurement module is configured to measure, automatically, the thoracic impedance at periodic intervals.
